(19) United States
(12) Patent Application Publication
(10) Pub. No.: US 2024/0251820 A1
(43) Pub. Date: Aug. 1, 2024

(54) IMPROVED SUGAR COATING PROCESS COMPRISING A HYDROLYZED AND FUNCTIONALIZED STARCH

(71) Applicant: ROQUETTE FRERES, Lestrem (FR)

(72) Inventors: Sébastien CROQUET, Merville (FR); Grégory LE BIHAN, CAMBRIN (FR); Philippe LEFEVRE, HAVERSKERQUE (FR); Claudia FRANCOIS, LESTREM (FR); Laurence CAMPION, LAMBERSART (FR)

(21) Appl. No.: 18/293,220

(22) PCT Filed: Jul. 29, 2022
(86) PCT No.: PCT/EP2022/025361

§ 371 Date: Jan. 29, 2024

(30) Foreign Application Priority Data
Jul. 30, 2021 (FR) 2108375

(51) Int. Cl. A23G 4/02; A23G 4/06; A23G 4/20; A61K 9/28

(52) U.S. Cl. CPC A23G 4/025; A23G 4/062; A23G 4/20; A61K 9/2826; A61K 9/2866; A61K 9/2893; A23G 2200/06; A23G 2220/20

(57) ABSTRACT

An improved sugar coating process using a hydrolyzed and functionalized starch. The present invention also relates to sugar-coating compositions comprising such a starch.

### TECHNICAL FIELD

The present invention relates to an improved sugar-coating method, as well as a sugar-coating composition useful for the implementation thereof. The present invention also relates to a solid form sugar-coated using this method or this composition.

### TECHNOLOGICAL BACKGROUND

Sugar coating is an operation used especially in confectionery or pharmaceutics, which consists in creating a more or less hard or soft crystallized coating on the surface of solid or powdery products, so as to protect them for various reasons or so as to make them attractive visually or in terms of flavor.

The coating of the solid form (core) is conventionally carried out in a tank rotating about its axis and called a coater, inside which there is a plurality of cores forming a moving mass, on the surface of which the constituent materials of the future envelope (“sugar-coating liquid or syrup”) is distributed in the liquid state. The hard and crystalline coating is obtained by applying this liquid and evaporating the water provided by it.

The sugar-coating syrup is mainly made up of one or even several crystallizable materials, and also conventionally contains binding agents such as gum arabic or gelatin, colorants, opacifiers such as TiO₂, mineral fillers such as talc, silica, calcium carbonate, intense sweeteners, flavorings, vitamins and active ingredients.

Sugar-coating is a relatively laborious method, including a large number of successive steps. Each of these steps, also called “sugar-coating cycles,” typically includes an application phase, generally by spraying a sugar-coating syrup onto the cores, a rotary phase for distributing said syrup onto the cores, also called pause time, and a phase of drying each new syrup layer produced by blowing hot and dry air.

Sugar-coating makes it possible to obtain solid shapes having a particularly attractive appearance, but lacks flexibility as to its implementation and as regards the visuals that can be obtained.

Interestingly, in patent application WO 20020/128290, the Applicant proposed an improved, particularly simple and flexible sugar-coating method for sugar-coating solid shapes. In addition, this method made it possible to sugar-coat solid forms comprising irregular shapes such as raised impressions or ridges, while retaining these irregular shapes. The crystallizable materials used were sugars or sugar alcohols such as xylitol, mannitol, sucrose, erythritol, isomalt or maltitol. The formulations could comprise binders such as gum arabic or polyvinyl alcohol (PVA). Gum arabic, contrary to PVA, has the advantage of being derived from renewable resources. However, it induces an unattractive yellowing of the sugar-coating layer.

There was thus a need to improve the formulations used in the above-mentioned method of patent application WO 20020/128290.

### OBJECTIVES

The present invention thus aims to provide sugar-coating compositions, in particular for use according to the method of the aforementioned patent application WO 20020/128290, which are composed of renewable materials.

The present invention also aims to provide sugar-coating compositions that do not yellow over time and/or that are free of gum arabic.

### PRESENTATION OF THE INVENTION

The Applicant has found that the use of a hydrolyzed and functionalized starch makes it possible to effectively replace the gum arabic in these compositions. In addition to the yellowing phenomenon which is avoided, the sugar-coated solid forms with a hydrolyzed and functionalized starch as a binder are equivalent or even superior to those sugar-coated with gum arabic as binder. In particular, the inventors noted that the hydrolyzed and functionalized starch used in accordance with the invention could make it possible to obtain improved stability of the sugar-coated solid forms.

### SUMMARY OF THE INVENTION

The first subject matter of the present invention is thus a sugar-coating method, comprising:
a step (a) of spraying a sugar-coating liquid comprising crystallizable materials onto a bed of moving cores, said cores being placed in a chamber equipped with a perforated rotary drum, and said spraying being performed by means of at least one compressed air nozzle;
at least one step (b) concomitant with step (a) of drying said sprayed sugar-coating liquid;
a step (c) of collecting the resulting sugar-coated solid forms;
   characterized in that said sugar-coating liquid comprises a hydrolyzed and functionalized starch.

Preferably, said crystallizable materials comprise xylitol.

Preferably, said hydrolyzed and functionalized starch is functionalized with alkyl groups having 1 to 5 carbon atoms.

Preferably, said hydrolyzed and functionalized starch is selected from hydroxypropylated starch, acetylated starch, hydroxyethyl starch, or a mixture thereof.

Another subject matter of the present invention is a sugar-coating liquid consisting of:
(a) 40 to 80% ingredients other than the solvent consisting of:
(a.1) 90.0 to 99.8% xylitol;
(a.2) 0.1 to 3.0% hydrolyzed and functionalized starch;
(a.3) 0.1 to 3.0% of an opacifier;
(a.4) 0.0 to 3.0% other ingredients;
the percentages (a.1) to (a.4) being expressed by weight relative to the total weight of ingredients other than the solvent, and their sum being equal to 100%; and,
(b) 60 to 20% solvent;
the percentages (a) and (b) being expressed by weight relative to the total weight of the sugar-coating liquid, and their sum being equal to 100%.

The present invention also relates to the use of such a sugar-coating liquid to sugar-coat solid forms.

The object of the present invention is also a sugar-coated solid form comprising at least one sugar-coating layer obtained from such a sugar-coating liquid.

A powdery composition for the sugar-coating of solid forms, consisting of:
90.0 to 99.8% xylitol;
0.1 to 3.0% hydrolyzed and functionalized starch;
0.1 to 3.0% of an opacifier;
0.0 to 3.0% other ingredients;
   the percentages being expressed by weight relative to the total weight of said powdery composition, and their sum being equal to 100%.

The present invention also relates to the use of such a powdery composition for sugar-coating solid forms.

The object of the present invention is also a sugar-coated solid form comprising at least one sugar-coating layer obtained from such a powdery composition.

### BRIEF DESCRIPTION OF THE DRAWINGS

FIG. 1 shows the maximum solubility of various crystallizable materials in water, based on the temperature of the solution.

### DESCRIPTION OF THE EMBODIMENTS

The invention thus firstly relates to a sugar-coating method, comprising:
a step (a) of spraying a sugar-coating liquid comprising crystallizable materials onto a bed of moving cores, said cores being placed in a chamber equipped with a perforated rotary drum, and said spraying being performed by means of at least one compressed air nozzle;
at least one step (b) concomitant with step (a) of drying said sprayed sugar-coating liquid;
a step (c) of collecting the resulting sugar-coated solid forms;
   characterized in that said sugar-coating liquid comprises a hydrolyzed and functionalized starch.

The expression “solid form” is conventionally understood to mean any solid presentation of sugar-coated substances (“sugar-coated solid form”) or capable of undergoing a sugar-coating operation (“core”). Typical examples are tablets, hard capsules, soft capsules, pellets, microspheres, granules, seeds, cookies, breakfast cereals, confectionery such as chewing gums, boiled candies, chewing candies, gummies, chocolates, fruits and vegetables, or products in the form of powders and/or crystals. These solid forms may, for example, be intended for food, pharmaceutical, veterinary or cosmetic use. They can be intended for humans, adults or children, or for animals. They may also be products intended for chemical or agrochemical use, although solid forms intended to be ingested (“solid oral forms”) in the context of the present invention are preferred. Preferably, these solid forms are selected from tablets and chewing gums.

The sugar-coating liquid of the invention comprises crystallizable materials and a functionalized hydrolyzed starch.

Conventionally, the term “crystallizable materials” in sugar-coating is understood to mean substances capable of crystallizing by evaporation of the solvent wherein they are dissolved. It is these crystallizable materials that form the crystalline coating targeted by the sugar-coating.

Preferably, the crystallizable materials of the sugar-coating liquid in accordance with the disclosure comprise at least one substance chosen from sugars and sugar alcohols, preferably chosen from monomers and dimers.

Preferably, in the sugar-coating liquid in accordance with the disclosure, the sugars and sugar alcohols represent at least 50%, preferably at least 70%, preferably at least 90% by weight of the crystallizable materials. Most preferentially, the crystallizable materials are entirely composed of substances chosen from sugars and sugar alcohols.

Preferably, these sugars and sugar alcohols are chosen from xylitol, sucrose, erythritol, mannitol, dextrose, isomalt, maltitol or optionally a combination thereof. Preferably, these crystallizable materials comprise xylitol. Preferably, the crystallizable materials of the sugar-coating liquid consist entirely of a single substance, preferably xylitol.

In a preferred embodiment, the sugar-coating method in accordance with the disclosure comprises:
a step (a) of spraying a sugar-coating liquid onto a bed of moving cores, said cores being placed in a chamber equipped with a perforated rotary drum, and said spraying being performed by means of at least one compressed air nozzle;
at least one step (b) concomitant with step (a) of drying said sprayed sugar-coating liquid;
a step (c) of collecting the resulting sugar-coated solid forms;
   characterized in that said sugar-coating liquid comprises xylitol and a hydrolyzed and functionalized starch.

The concentration of crystallizable materials of the sugar-coating liquid is chosen so that the crystallizable materials are solubilized in said sugar-coating liquid. Typically, in particular when the sugar-coating liquid is an aqueous liquid, the sugar-coating liquid in accordance with the disclosure has a concentration of crystallizable materials less than or equal to 90%, this percentage being expressed by weight relative to the total weight of said sugar-coating liquid. Preferably, this concentration is less than or equal to 80%, preferably less than or equal to 70%. This concentration is preferably greater than or equal to 20%, preferably greater than or equal to 30%, preferably greater than or equal to 40%, preferably greater than or equal to 50%. It is for example equal to about 60%.

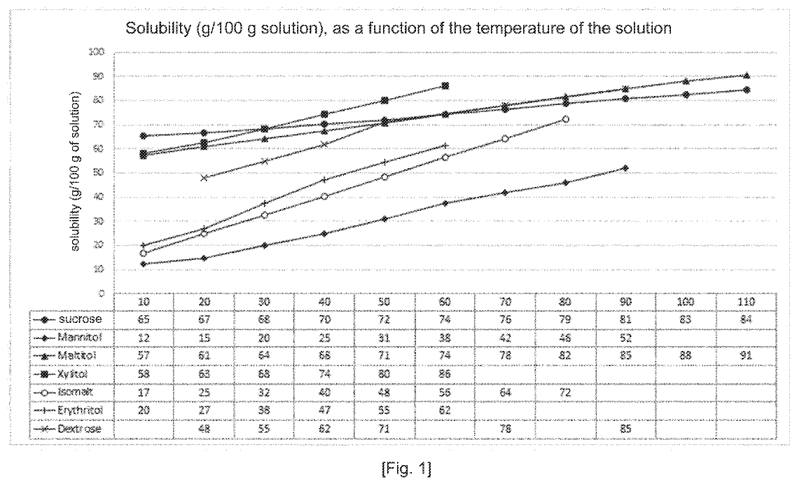

The maximum concentration of crystallizable materials naturally depends on the nature of these crystallizable materials, as well as on the temperature of the sugar-coating liquid. For xylitol, sucrose, erythritol, mannitol, dextrose, isomalt, and maltitol, reference may for example be made to FIG. 1, which exhibits the maximum concentrations of these crystallizable materials in water based on the temperature of the solution. Thus, for example, the concentration of crystallizable materials of a sugar-coating liquid at 20° C. is preferentially less than or equal to 67% when the composition of crystallizable materials consists of sucrose, less than or equal to 15% when it is mannitol, less than or equal to 61% when it is maltitol, less than or equal to 63% when it is xylitol, less than or equal to 25% when it is isomalt, less than or equal to 27% when it is erythritol, less than or equal to 48% when it is dextrose.

Preferably, the content of crystallizable materials or of xylitol of the sugar-coating liquid is greater than or equal to 50% by weight relative to the total weight of ingredients other than the solvent of the sugar-coating liquid. This content is preferably greater than or equal to 70%, preferably greater than or equal to 80%, preferably greater than or equal to 90%, preferably greater than or equal to 95%, for example equal to about 99%.

Preferably, the sugar-coating liquid in accordance with the disclosure has a content of crystallizable materials, in particular of xylitol, of less than or equal to 80%, preferably of less than or equal to 70%, preferably of less than or equal to 60%, this percentage being expressed by weight relative to the total weight of the sugar-coating liquid. This content is preferably greater than or equal to 20%, preferably greater than or equal to 30%, preferably greater than or equal to 40%, preferably greater than or equal to 50%. It is for example equal to about 59%.

The sugar-coating liquid of the invention further has a hydrolyzed and functionalized starch.

It is recalled that the expression “starch” conventionally refers to the starch isolated from any suitable botanical source, by any technique well known to a person skilled in the art. The isolated starch generally does not contain more than 3% of impurities; said percentage being expressed by dry weight of impurities relative to the total dry weight of isolated starch. These impurities typically comprise proteins, colloidal materials and fibrous residues. An appropriate botanical source comprises, for example, leguminous plants, cereals and tubers. The starches in accordance with the disclosure may be derived, for example, from leguminous plants (for example, pea), cereals (for example, corn, rice, wheat, oats) and tubers (for example potato, tapioca).

Preferably, the hydrolyzed and functionalized starch in accordance with the disclosure is derived from a leguminous starch and/or cereals, preferably pea or corn starch, preferably pea, preferably smooth pea. In a preferred embodiment, the hydrolyzed and functionalized starch in accordance with the disclosure, in particular when it is derived from a pea starch, is derived from a starch having an amylose content of 20 to 50%, preferably from 30 to 45%, preferably from 30 to 40%, for example equal to about 35%, these percentages being expressed by dry weight of amylose relative to the total dry weight of starch. This amylose content can be determined by a person skilled in the art by potentiometric analysis of the iodine absorbed by the amylose to form a complex. In another preferred embodiment, the hydrolyzed and functionalized starch in accordance with the disclosure, in particular when it is derived from a corn starch, is derived from a starch having an amylose content greater than 5%, preferably from 10 to 40%, preferably from 15 to 35%, preferably from 20 to 30%, for example equal to about 25%.

The hydrolyzed and functionalized starches in accordance with the disclosure can be hydrolyzed by any appropriate technique, for example by acid treatment, heat treatment, enzymatic treatment or a combination thereof. They may be dextrins, comprising maltodextrins or pyrodextrins. “Maltodextrins” conventionally refer to hydrolyzed starches having a dextrose equivalent of 2 to 20. They are generally obtained by acid or enzymatic hydrolysis. “Pyrodextrins” are conventionally obtained from the action of high temperatures (generally of at least 100° C.), combined or not with the action of an acid or a base, in the presence of small quantities of water. Such a treatment conventionally leads to branches and to the formation of so-called “atypical” bonds, and pyrodextrins are therefore structurally different from the other hydrolyzed starches, in particular maltodextrins. Preferably, the hydrolyzed starches in accordance with the disclosure are obtained by acid hydrolysis. Preferably, the functionalized hydrolyzed starches in accordance with the disclosure are not pyrodextrins.

Hydrolyzed starches must be well differentiated from glucose, glucose syrups and cyclodextrins, whose hydrolysis rate is too high to also be called “starches” as conventionally understood by the person skilled in the art. In other words, glucose, glucose syrups and cyclodextrins are not hydrolyzed starches within the meaning of the invention.

The functionalized starch according to the invention being hydrolyzed, this means that it has a reduced molecular weight compared to the native starch from which it is derived. Therefore, alternatively or additionally, the hydrolyzed and functionalized starch in accordance with the disclosure may be defined by its weight average molecular weight (Mw), which is preferably less than 10,000 kDa as determined by size exclusion chromatography (HPSEC) with refractometric detection (RI) and coupled with light scattering (MALLS). Preferably, the Mw is determined by the following method: samples of hydrolyzed and functionalized starch are diluted in a mixture of dimethylsulfoxide and 0.1 M sodium nitrate. Two columns (for example, SUPREMA columns) having a porosity of 100 and 1000 Angstrom are used. The elution is carried out in an aqueous medium at a flow rate of 0.5 mL/min. The system is maintained at a temperature of 40° C. Preferably, the hydrolyzed and functionalized starch in accordance with the disclosure has an Mw less than or equal to 9,000 kDa, preferably less than or equal to 7,000 kDa, preferably less than or equal to 5,000 kDa, preferably less than or equal to 4,000 kDa. It is preferably greater than or equal to 100 kDa, preferably greater than or equal to 500 kDa, preferably greater than or equal to 1,000 kDa, preferably greater than or equal to 2,000 kDa, preferably greater than or equal to 2,500 kDa, preferably greater than or equal to 3,000 kDa. It is for example equal to about 3,500 kDa.

Preferably, the hydrolyzed and functionalized starch in accordance with the disclosure has a viscosity in water of less than 10,000 mPa·s at 20° C., at a shear rate of 100 s−1, determined on an aqueous solution comprising 10% dry weight of said starch. Preferably, the viscosity is determined on a rheometer (for example, Physica MCR301, Anton Paar) with a 5 cm 1° (CP50) cone-plate geometry. It is possible, for example, to proceed as follows: the temperature is regulated, for example, via Peltier. The 10% dry starch solution is maintained at equilibrium at 20° C. Shear rates ranging from 0.006 to 1000 s⁻¹ (log) are applied in 3 minutes at 20° C. Preferably, this viscosity (at 10% dry; 20° C.; a shear rate of 100 s⁻¹) is less than or equal to 5,000 mPa·s, preferably less than or equal to 1,000 mPa·s, preferably less than or equal to 500 mPa·s, preferably less than or equal to 100 mPa·s, preferably less than or equal to 50 mPa·s. It is preferably greater than or equal to 1 mPa·s, preferably greater than or equal to 10 mPa·s, preferably greater than or equal to 20 mPa·s, preferably greater than or equal to 30 mPa·s, for example equal to about 40 mPa·s. It is for example equal to about 42 mPa·s.

The hydrolyzed starch in accordance with the disclosure is also functionalized. In other words, it comprises functional groups grafted to at least one of its hydroxyl functions. In other words, the expression “functionalized” within the meaning of the invention excludes crosslinking. Preferably, the functionalized starch in accordance with the disclosure is etherified and/or esterified, preferably etherified. Preferably, the starch in accordance with the disclosure is functionalized with alkyl groups having 1 to 5 carbon atoms, preferably 2 to 4 carbon atoms, preferably 2 or 3 carbon atoms, preferably 3 carbon atoms. Preferably, the alkyl group is an aliphatic alkyl group, more preferably a saturated aliphatic group.

Preferably, the functionalized starch in accordance with the disclosure is chosen from hydroxypropylated starch, acetylated starch, hydroxyethyl starch, or a mixture thereof. It is preferably hydroxypropylated starch, preferably used alone.

The hydroxypropylated starch in accordance with the disclosure can be obtained for example by etherification with propylene oxide. The hydrolyzed hydroxypropylated starch in accordance with the disclosure preferably has a content of hydroxypropyl groups of 0.5 to 10%; said percentage being expressed in dry weight of hydroxypropyl groups relative to the total dry weight of hydrolyzed hydroxypropylated starch. The content of hydroxypropyl groups can be determined by a person skilled in the art, for example by proton Nuclear Magnetic Resonance (proton NMR), preferably according to a method in accordance with the European Pharmacopeia (“STARCH, PREGELATINIZED HYDROXYPROPYL”) as in force on 1st 2021. It is preferably from 0.5 to 9%, preferably from 0.5 to 8%, preferably from 0.5 to 7%, for example equal to about 7%.

The functionalized starch in accordance with the disclosure may be etherified or esterified or both etherified and esterified. However, it is preferably either esterified or etherified. It may be substituted (that is, functionalized) with groups of the same nature or of different natures. However, it is preferably substituted with groups of the same nature, for example and preferably substituted only by hydroxypropyl groups.

Preferably, the hydrolyzed and functionalized starch in accordance with the disclosure has a cold solubility in water equal to or greater than 70%, said percentage being expressed by dry weight of soluble starch relative to the total weight of starch. Preferably, this cold solubility in water is greater than or equal to 80%, preferably greater than or equal to 90%, preferably greater than or equal to 95%. It is for example equal to about 98%. This solubility can be determined for example by placing 5 grams of said starch in 200 mL of distilled water, at 20° C. The dissolved dry weight can be determined after centrifuging, and desiccation of the supernatant. This solubility may for example be determined according to the following protocol: in a 250 ml beaker, 200 mL of distilled water is added. 5 g of starch is added and the mixture is homogenized by magnetic stirring for 15 minutes. The resulting solution/suspension is centrifuged for 10 minutes at 4,000 rpm. 25 mL of the supernatant is taken and introduced into a crystallizer and placed in an oven at 60° C., until the water is evaporated. It is next placed in an oven at 103ºC+2° C. for 1 hour. The residue is placed in a desiccator for cooling to room temperature and then weighed in order to calculate the solubility by dry weight.

A starch having such a cold solubility in water can be conventionally obtained from insoluble starch by firing and/or hydrolysis of said insoluble starch. Said firing can typically be carried out by heating a starch suspension, so that the insoluble granules burst and dissolve. The functionalized starch in accordance with the disclosure being hydrolyzed, it is sometimes cold soluble in water without requiring additional treatment, according of course to the level of hydrolysis.

Preferably, the hydrolyzed and functionalized starch in accordance with the disclosure is pregelatinized. Pregelatinization conventionally means that the starch particles no longer exhibit birefringence (absence of crystalline phase) with a polarized light optical microscope. Pregelatinization can be commonly obtained from birefringent starch, via a heat treatment (50-90° C. in general, in particular based on the botanical origin of the starch) in the presence of water (said treatment commonly being called “firing,” as in the preceding paragraphs) with \ additional drying (after firing and/or concomitantly). Chemical substances can be used as processing aids. The pregelatinization can be carried out for example by drum-drying. In this case, the starch can be cured before or during the drum-drying step. It can also be fired and then atomized. It also can be obtained by extrusion. In the present disclosure, the pregelatinization is preferentially carried out by firing and atomization.

The hydrolyzed and functionalized starch in accordance with the disclosure may undergo other chemical and/or physical modifications than the preferred ones described above, so long as the latter do not contradict the properties sought here, in particular concerning the quality of the sugar-coated solid forms obtained and/or the workability of the method. However, and because it appears that this is not necessary to address the problem disclosed herein, the hydrolyzed and functionalized starch in accordance with the disclosure is preferably not further modified.

Preferably, the hydrolyzed and functionalized starch in accordance with the disclosure is a product of CAS No. 9049-76-7. Preferably, the hydrolyzed and functionalized starch in accordance with the disclosure is in accordance with the European Pharmacopeia (“STARCH, PREGELATINIZED HYDROXYPROPYL”) in force on Mar. 1, 2021.

Particularly useful functionalized hydrolyzed starches are commercially available. Mention may be made, for example, of hydrolyzed hydroxypropylated starches LYCOAT® RS 720 (CAS No. 113894-92-1) or LYCOAT® RS 780 (CAS No. 113894-92-1) sold by the Applicant.

Preferably, the functionalized hydrolyzed starch content of the sugar-coating liquid is greater than or equal to 0.1%, preferably greater than or equal to 0.2%, preferably greater than or equal to 0.3%, preferably greater than or equal to 0.4%, this percentage being expressed in weight relative to the total weight of ingredients other than the solvent of the sugar-coating liquid. This content is preferably less than 5.0%, preferably less than or equal to 4.0%, preferably less than or equal to 3.0%, preferably less than or equal to 2.0%, preferably less than or equal to 1.0%. It is for example about 0.1 to about 1.0%, or about 0.5 to about 0.9%.

Preferably, the crystallizable material/functionalized hydrolyzed starch weight ratio is greater than or equal to 50, preferably greater than or equal to 70, preferably greater than or equal to 90, preferably greater than or equal to 100, preferably greater than or equal to 110, preferably greater than or equal to 120, preferably greater than or equal to 130, preferably greater than or equal to 140, preferably greater than or equal to 150, preferably less greater than or equal to 170, preferably greater than or equal to 190. It is preferably less than or equal to 300, preferably less than or equal to 250, preferably less than or equal to 230, preferably less than or equal to 210. It is for example equal to about 200.

Preferably, the sugar-coating liquid in accordance with the disclosure has a content of ingredients other than solvent of less than or equal to 80%, preferably of less than or equal to 70%, preferably of less than or equal to 65%, this percentage being expressed in weight relative to the total weight of the sugar-coating liquid. This content is preferably greater than or equal to 20%, preferably greater than or equal to 30%, preferably greater than or equal to 40%, preferably greater than or equal to 50%, preferably greater than or equal to 55%. It is for example equal to about 60%.

The sugar-coating liquid according to the disclosure is typically polar, and preferentially comprises water as the majority solvent, most preferentially as the sole solvent.

Preferably, for reasons of simplicity and since this is possible, the sugar-coating method according to the invention implements a single sugar-coating liquid, that is, the sugar-coating liquid has a constant formulation throughout the duration of the sugar-coating.

The sugar-coating liquid according to the disclosure may comprise substances other than those previously mentioned, as long as this does not contravene the properties sought herein, in particular related to the quality of the sugar-coated solid forms obtained and/or the workability of the method. Such other compounds are, for example:
other binders, such as gum arabic, polyvinyl alcohol;
crystallizable materials and/or sugars and/or sugar alcohols other than xylitol;
colorants such as pigments;
flavorings, sweeteners;
active agents of interest, for example, pharmaceutical, nutritional, nutraceutical or phytosanitary agents.

However, preferably, in the sugar-coating liquid in accordance with the disclosure, the functionalized hydrolyzed starch represents at least 50% by weight of the binding agents, preferably at least 60%, preferably at least 70%, preferably at least 80%, preferably at least 90%, preferably about 100%. Thus preferably, the functionalized hydrolyzed starch in accordance with the disclosure is the only binder of the sugar-coating liquid. In particular, the sugar-coating liquid in accordance with the disclosure is preferably free of PVA or free of gum arabic, preferably free of PVA and gum arabic.

The crystallizable materials other than xylitol are conventionally selected from sugars and sugar alcohols, conventionally selected from monomers and dimers, preferably from mannitol, sucrose, erythritol, dextrose, isomalt, maltitol or optionally a combination thereof. Preferably, in the sugar-coating liquid in accordance with the disclosure, xylitol represents at least 50% by weight of the sum of the crystallizable materials and/or the sum of the sugars and sugar alcohols. Preferably, this xylitol content is greater than or equal to 60%, preferably greater than or equal to 70%, preferably greater than or equal to 80%, preferably greater than or equal to 90%, preferably equal to about 100%. Thus preferably, the sugar-coating liquid in accordance with the disclosure is free of crystallizable materials other than xylitol and/or free of sugars, and/or free of sugar alcohols other than xylitol.

Preferably, the sugar-coating liquid in accordance with the disclosure further comprises an opacifier, preferably titanium dioxide. Preferably, the functionalized opacifier content of the sugar-coating liquid is greater than or equal to 0.1%, preferably greater than or equal to 0.3%, preferably greater than or equal to 0.5%, preferably greater than or equal to 0.7%, preferably greater than or equal to 0.9%, this percentage being expressed in weight relative to the total weight of ingredients other than the solvent of the sugar-coating liquid. Preferably, this opacifier content, in particular of titanium dioxide, is less than or equal to 20.0%, preferably less than or equal to 15.0%, less than or equal to 10.0%, less than or equal to 5.0%. This content is for example equal to about 1%.

Generally, the sugar-coating liquid in accordance with the disclosure comprises an amount of less than 5.0% of pigments and/or colorants, preferably less than or equal to 4.0%, preferably less than or equal to 3.0%, preferably less than or equal to 2.0%, preferably less than or equal to 1.0%, for example equal to about 0.0%, these percentages being expressed by weight relative to the total weight of ingredients other than the solvent of the sugar-coating liquid.

In another preferred embodiment, the sugar-coating liquid in accordance with the disclosure consists of:
(a) from 40 to 80%, preferably from 45 to 75%, preferably from 50 to 70%, preferably from 55 to 65%, for example about 60%, of ingredients other than the solvent consisting of:
(a.1) from 90.0 to 99.8% of xylitol, preferably from 95.0% to 99.5%, preferably from 96.0% to 99.0%, preferably from 97.0% to 99.0%, preferably from 98.0% to 99.0%, for example 98.5%;
(a.2) from 0.1 to 3.0% of hydrolyzed and functionalized starch, preferably from 0.2 to 2.0%, preferably from 0.3 to 1.0%, preferably from 0.4 to 0.8%, preferably from 0.4 to 0.7%, preferably from 0.4 to 0.6%, for example 0.5%;
(a.3) from 0.1 to 3.0% of opacifier, preferably from 0.5 to 2.0%, for example 1.0%;
(a.4) from 0.0 to 3.0% of other ingredients, preferably colorant substances, preferably less than 2.0%, preferably less than 1.0%, preferably less than 0.5%, for example 0.0%;
the percentages (a.1) to (a.4) being expressed by weight relative to the total weight of ingredients other than the solvent, and their sum being equal to 100%; and
(b) from 60 to 20%, preferably from 55 to 25%, preferably from 50 to 30%, preferably from 45 to 35%, for example approximately 40% of solvent, said solvent preferentially being water;
the percentages (a) and (b) being expressed by weight relative to the total weight of the sugar-coating liquid, and their sum being equal to 100%.

The present invention also relates to the use of such a sugar-coating liquid for sugar-coating solid forms. Preferably, said sugar-coating is carried out according to the method in accordance with the disclosure as described before.

The sugar-coating method according to the disclosure may further comprise a step of preparing the sugar-coating liquid, preferably by mixing a ready-to-use mixture with a suitable solvent, preferably water.

Preferably, this ready-to-use mixture has the composition as defined previously for the sugar-coating liquid, with the exception of the solvent, which is of course omitted.

The ready-to-use mixture in accordance with the disclosure is typically in the form of a powdery mixture.

In a preferred embodiment, the ready-to-use mixture in accordance with the present disclosure is a powdery composition consisting of:
from 90.0 to 99.8% of xylitol, preferably from 95.0% to 99.5%, preferably from 96.0% to 99.0%, preferably from 97.0% to 99.0%, preferably from 98.0% to 99.0%, for example 98.5%;
from 0.1 to 3.0% of hydrolyzed and functionalized starch, preferably from 0.2 to 2.0%, preferably from 0.3 to 1.0%, preferably from 0.4 to 0.8%, preferably from 0.4 to 0.7%, preferably from 0.4 to 0.6%, for example 0.5%;
from 0.1 to 3.0% of opacifier, preferably from 0.5 to 2.0%, for example 1.0%;
from 0.0 to 3.0% of other ingredients, preferably colorant substances, preferably less than 2.0%, preferably less than 1.0%, preferably less than 0.5%, for example 0.0%;
the percentages being expressed by weight relative to the total weight of said powdery composition, and their sum being equal to 100%.

The present invention also relates to the use of such a powdery composition for sugar-coating solid forms. Preferably, said sugar-coating is carried out according to the method in accordance with the disclosure as described before.

The equipment used to implement the method according to the disclosure typically comprises a unit for storing the sugar-coating liquid, comprising at least one outlet for transporting the sugar-coating liquid to a device for spraying the sugar-coating liquid. This transport can for example be ensured by means of a peristaltic pump. The sugar-coating liquid is applied, via the spraying device, to a bed of cores contained in the chamber, said chamber being provided with a rotary drum for moving said bed of cores. More precisely, the drum is a perforated rotary drum, and the selected spraying device comprises at least one compressed air nozzle. The equipment further comprises an air inlet at the drum chamber for drying the sugar-coating liquid. The drying air is discharged in particular via the perforations of the rotary drum, in particular by suction of the air from the chamber.

The elements of the equipment useful for the method according to the disclosure are commercially available, and their arrangement does not represent particular difficulties for the person skilled in the art.

The temperature of the sugar-coating liquid according to the disclosure is typically selected so that the crystallizable materials, in particular xylitol, are well solubilized in said sugar-coating liquid to be sprayed. This temperature is thus also a function of the amount of crystallizable materials present in the liquid. Preferably, the temperature of the sugar-coating liquid is less than 90° C., preferably less than 80° C., preferably less than 70° C., preferably less than 60° C., preferably less than 50° C., preferably less than 40° C., preferably less than 30° C. This temperature is preferably at least 10° C., preferably at least 15° C., or even at least 20° ° C. It preferably corresponds to ambient temperature, which typically varies from about 20 to about 25° C. When xylitol is used as crystallizable material, the temperature of the solvent used for the sugar-coating liquid is preferably as defined above in this paragraph. However, the temperature of the sugar-coating liquid will generally be lower, due to the high dissolution enthalpy of xylitol. Thus, for example, for a sugar-coating liquid using water at 20° C. as solvent, the sugar-coating liquid will generally have a temperature of 10 to 15° C. after dissolution of the xylitol in water, and from 15 to 20° C. during the sugar-coating.

Preferably, the sugar-coating liquid is kept under stirring during its use. Preferably, the rotational speed is chosen so as to maintain the homogeneity of the sugar-coating liquid.

In a preferred embodiment, the sugar-coating liquid is stored in a single jacket storage unit, and/or the equipment useful in the method according to the disclosure does not have a device for heating the sugar-coating liquid. Indeed, since the method according to the disclosure does not necessarily require the use of high temperatures of sugar-coating liquids, these devices are not obligatory in the method according to the disclosure.

For spraying the sugar-coating liquid, the number of compressed air nozzles used is conventionally selected based on the dimensions of the sugar-coating chamber, according to the manufacturer's recommendations. This number of nozzles is typically from 1 to 2 nozzle(s) per 40 cm diameter section of the sugar-coating chamber. This number of nozzles ranges, for example, from 1 to 10, for example from 1 to 6.

Preferably, for spraying, the method according to the disclosure uses only compressed air nozzles.

Preferably, the nozzles used in accordance with the disclosure have a hole having a diameter selected from a range from 0.1 to 2.8 mm, preferably from 0.1 to 2.5 mm, preferably from 0.1 to 2.2 mm, for example from 0.3 to 2.0 mm, or from 0.5 to 1.8 mm or from 0.5 to 1.5 mm, or from 0.5 to 1.2 mm, or from 0.5 to 1.0 mm.

Preferably, the spray flow rates are selected from a range from 1 to 20 g/min/kg of cores, preferably from 1 to 15 g/min/kg of cores, preferably from 2 to 15 g/min/kg of cores, preferably from 5 to 10 g/min/kg of cores. Preferably, the flow rate selected for the spraying is increased during the sugar-coating method. In particular, the inventors have found that, for certain crystallizable materials, the use of low flow rates at the beginning of sugar-coating makes it possible to promote the first crystallization phase which takes place on the surface of the cores. The flow rate can then be increased so as to accelerate the sugar-coating.

For spraying, the atomization pressure and collapse pressure are adjusted based on the flow rate and the hole of the nozzle, and in accordance with the manufacturer's recommendations. This flow rate and this nozzle hole typically depend on the size of the equipment used. These atomization and collapse pressures are typically selected in a range from 0.5 to 4.0 bar, preferably from 0.5 to 3.5 bar, for example from 0.7 to 2.5 bar for the atomization pressure, and/or from 0.7 to 3.5 bar for the collapse pressure. The atomization pressure is for example equal to about 1.

In the sugar-coating method of the invention, the sugar-coating liquid is sprayed onto a bed of cores moved by means of a rotary drum. The nature of these cores is preferably as defined above for solid forms; they are, for example, tablets or chewing gums. These cores may be completely bare, or may be coated with one or more layers, for example gumming, film-coating or even sugar-coating layers, said layers preferentially being obtained in the same equipment as that used for the sugar-coating method according to the disclosure.

To set the bed of cores in motion, the speed of rotation of the drum is selected based on the dimensions of the chamber and of the size of the cores to be sugar coated. It is generally selected in a range from 3 to 30 rpm, preferably in a range from 5 to 20 rpm, preferably in a range from 5 to 15 rpm. It is for example equal to about 9 rpm, in particular for a perforated rotary drum of 48.26 cm in diameter.

In an advantageous embodiment, in particular for reasons of simplicity and/or space, and because the method according to the disclosure allows it, the moving of the bed of cores excludes the transport of said cores along a longitudinal axis. This means in particular that the cores being sugar-coated are not transported from one chamber to another, that is, they are sugar-coated in a single chamber, and/or that the bed of cores is not sugar-coated in a longitudinal chamber along which they are transported.

For the drying, the inlet air temperature chosen is preferably less than 100° C., preferably less than or equal to 80° C., preferably less than or equal to 70° C., preferably less than or equal to 60° C., preferably less than or equal to 50° C. This temperature is generally greater than or equal to 20° C., preferably greater than or equal to 30° C. It is for example equal to about 40° C.

For drying, the air flow rate may be selected in a range from 50 to 8000 m³/h, for example, in a range from 100 to 7000 m³/h, for example, from 100 to 1000 m³/h. This volume is, for example, equal to 500 m³. It generally depends on the size of the turbine.

The outlet of the drying air is preferably carried out by suction of air by means of the perforations of the perforated rotary drum.

Preferably, the perforated wall area of the rotary drum according to the disclosure represents at least 50% of the surface of said wall of the drum, preferably at least 60%, preferably at least 70%, preferably at least 80%, preferably at least 90%. Also preferably, the wall of the drum is perforated over its entire surface.

Preferably, the temperature of the bed of cores being sugar-coated is less than or equal to 70° C., preferably less than or equal to 60° C., preferably less than or equal to 50° C., preferably less than or equal to 40° C. This temperature of the bed of cores during sugar-coating is generally at least 10° C., or even at least 20° C. This temperature of the bed of cores being sugar-coated is for example about 25 to about 30° C. Preferably, before the start of the sugar-coating, that is, before the spraying begins, the sugar-coating method in accordance with the disclosure comprises a step of heating the bed of cores to be sugar-coated. This step aims in particular at bringing the core bed to a target temperature which corresponds to that of the cores being sugar-coated.

The sugar-coating method of the invention concomitantly sprays and dries the spraying liquid. However, it is conceivable to introduce distribution (“pause time”) and spraying steps in the absence of drying, as long as this does not contravene the properties sought herein, in particular relating to the quality of the sugar-coated solid forms obtained and/or the workability of the method.

Preferably, the phases of the sugar-coating method, during which spraying is optionally not carried out concomitantly with drying, represent less than 50% of the sugar-coating method time, preferably less than 40%, preferably less than 30%, preferably less than 20%, preferably less than 10%, preferably less than 5%. Most preferentially, the sugar-coating does not comprise a spraying phase in the absence of drying.

Preferably, the possible pause times (time between two spraying phases during which there is neither spraying nor drying) represent less than 50% of the time of the sugar-coating method, preferably less than 40%, preferably less than 30%, preferably less than 20%, preferably less than 10%, preferably less than 5%. Most preferentially, the sugar-coating does not comprise a pause time.

Preferentially, the percentage of sugar-coating (or “mass gain”) is, per unit of time, at least 0.1% per minute, preferably at least 0.2% per minute, preferably at least 0.3% per minute, preferably at least 0.4% per minute. This mass gain per unit of time is generally less than 1.0% per minute, or even less than 0.8% per minute, or even less than 0.7% per minute. It is for example equal to about 0.4 or 0.5% per minute.

The method according to the disclosure may further comprise other usual steps than those aimed at sugar-coating the solid forms, as long as this does not contravene the properties sought herein, in particular related to the quality of the sugar-coated solid forms obtained and/or the workability of the method. Examples of other steps which may be mentioned include gumming, smoothing, polishing and coloring. Advantageously, if such steps are carried out, they are carried out in the same equipment as that used in the sugar-coating method according to the disclosure. It is understood that some of these steps, such as, for example, gumming or coloring, may sometimes be assimilated to sugar-coating steps, if the composition used comprises such an amount of crystallizable materials that a layer of crystallized substances, in particular sugars and/or sugar alcohols, is effectively formed.

Preferably, the sugar-coating method in accordance with the disclosure comprises a smoothing step, after the sugar-coating. Preferably, this smoothing step uses the sugar-coating liquid in accordance with the disclosure for its implementation. Preferably, this smoothing comprises a first step of spraying the liquid without drying, a second step without spraying or drying, a third step without spraying but with drying. These three steps make up a smoothing cycle according to a preferential embodiment. Preferably, the smoothing comprises 1 to 5 cycles, preferably 1 to 4, preferably 1 to 3.

The coloring, if it is desired, may be carried out directly via adding coloring agents in the sugar-coating liquid.

The equipment useful for the method according to the disclosure may comprise other elements conventionally used in sugar-coating, as long as this does not contravene the properties sought herein, in particular related to the quality of the sugar-coated solid forms obtained and/or the workability of the method. Preferably, however, the chamber of the drum used in accordance with the disclosure is free of lump breakers.

The present invention also relates to a sugar-coated solid form obtained or capable of being obtained according to the sugar-coating method in accordance with the disclosure. The present invention also relates to a sugar-coated solid form using a sugar-coating liquid in accordance with the disclosure.

Preferably, the sugar-coated solid forms according to the disclosure have a percentage of sugar-coating greater than 1%. This percentage of sugar-coating, also known as “mass gain”, is conventionally determined as follows:

$$\% sugar - coating = \frac{Sugar - coated solid form weight - Core weight}{Core weight} \times 100 \qquad [ Math . 1 ]$$

Preferably, this percentage of sugar-coating is greater than 3%, preferably greater than 5%, preferably greater than 10%, preferably greater than or equal to 20%, preferably greater than or equal to 25%. It is preferably less than or equal to 50%, preferably less than or equal to 40%, preferably less than or equal to 35%. It is for example equal to about 30%.

In the present disclosure, the quantities of ingredients are generally expressed as percentages by weight. Unless otherwise indicated, these weights are quantities of ingredients in their original form, which is generally in powder form. These powdery ingredients generally contain small quantities of water (also called % moisture or “mass loss with desiccation”) and some impurities. In this respect, hydrolyzed and functionalized starch in accordance with the disclosure generally contains no more than 15% by weight of water, generally 3 to 12%; sugar alcohols such as xylitol generally contain no more than 1% water. This means that when for example reference is made to 10% by weight of hydrolyzed and functionalized starch, this generally corresponds to 8.8 to 9.7% by dry weight (that is, by anhydrous weight).

In contrast, in the present disclosure, when reference is made to a dry weight, this refers to anhydrous weights.

Other features and advantages of the present invention will become clearly apparent on reading the examples below, which illustrate the invention without, however, limiting it.

### Examples

### A. Sugar-Coating Tests

### 1. Equipment Used

Equipment with the following characteristics was used:
Perforated rotary drum: 48.26 cm diameter;
Blades ×6;
1 compressed air atomization nozzle (Schlick 970/7-1 S75); 0.8 mm hole diameter;
Peristaltic pump (Watson Marlow model 323); 3 roller head (313 DW);
Exacanal Tube: 4 mm internal diameter: 8 mm external diameter.

### 2. Cores

Chewing gums having the following qualitative composition were used: sorbitol (NEOSORBR P60W, ROQUETTE), base gum, maltitol syrup (LYCASIN® 85/55, ROQUETTE), mannitol (Mannitol 60, ROQUETTE), powder flavoring, liquid flavoring, sucralose, acesulfame K)

### 3. Common Parameters for all Tests

TABLE 1

| Mass of cores to be sugar- | 5 | kg |
|---|---|---|
| coated | | |
| Operating pressure | 4 | bar |
| Atomization pressure | 1.2 | bar |
| Jet collapse pressure | 1.2 | bar |
| Air flow rate | 500 | m³/h |
| Differential pressure of the | Not known, but less than 0 | |
| atomization chamber | | |
| Rotational speed | 9 | rpm |
| Inlet air temperature | 40° | C. |

4. Sugar-Coating with Xylitol and Gum Arabic

The operating conditions were optimized so as to obtain the best results with gum arabic. The best results were obtained under the conditions indicated in this section.

The sugar-coating liquid had the following formulation:

TABLE 2

| | |
|---|---|
| Xylitol (XYLISORB ®90, ROQUETTE) | 58.7% |
| Gum arabic (NOREVO) (binder) | 0.3% |
| Titanium dioxide (KRONOS) | 0.6% |
| Sum of ingredients other than the solvent | 59.6% |
| Demineralized water (solvent) | 40.4% |

The percentages are expressed by weight relative to the total weight of the sugar-coating liquid.

The sugar-coating liquids were prepared as follows: two solutions were prepared. A solution A was prepared by mixing water with xylitol in a blade mixer. The gum arabic was then introduced in the form of a 40% solution. The whole was stirred for about 45 minutes at a rate sufficient to disperse and dissolve the xylitol. A solution B comprising titanium dioxide and water was prepared in a high shear mixer (POLYTRON) (3-5 minutes, 11,000-30,000 rpm). Solution A was added to solution B.

The sugar-coating conditions were as follows:

TABLE 3

| | Spraying flow | Spraying flow | Core bed | |
|---|---|---|---|---|
| Pump | rate over 5 kg | rate per kg | temperature | Time |
| (rpm) | (g/min/5 kg) | (g/min/kg) | (° C.) | (min) |
| 16 | 30 | 6 | 30 | 0->5 |
| 22 | 40 | 18 | 28 | 5->56 |

A smoothing step was then carried out as follows, using the same solution as that used for the sugar-coating liquid.

TABLE 4

| | | | Time | | |
|---|---|---|---|---|---|
| | Amount of | | without | Time | |
| | solution | Spraying | spraying | without | |
| | nebulized | time | and | spraying | Total time |
| Number of | without | without | without | and with | of the |
| smoothing | drying | drying | drying air | drying air | smoothing |
| steps | air (g) | air (s) | (min) | (min) | step (min) |
| 1 | 60* | 40 | 2.5 | 0.5 | 8 |
| 1 | 50* | 35 | 2.5 | 0.5 | |

Pump speed: 50 rpm

5. Sugar-Coating with Xylitol and Functionalized Hydrolyzed Starch

The operating conditions were optimized so as to obtain the best results with a functionalized hydrolyzed starch in accordance with the disclosure.

### Test 1

The sugar-coating liquid had the following formulation:

TABLE 5

| | |
|---|---|
| Xylitol (XYLISORB ®90, ROQUETTE) | 58.7% |
| Hydrolyzed and functionalized starch | 0.2% |
| (hydrolyzed hydroxypropylated starch | |
| LYCOAT ® RS720, ROQUETTE) (binder) | |
| Titanium dioxide (KRONOS) | 0.6% |
| Sum of ingredients other than the solvent | 59.5% |
| Demineralized water (solvent) | 40.5% |

The percentages are expressed by weight relative to the total weight of the sugar-coating liquid.

The sugar-coating liquids were prepared as in point 4. LYCOAT® RS720 was introduced in the form of a 25% solution.

The sugar-coating conditions were as follows:

TABLE 6

| | Spraying flow | Spraying flow | Core bed | |
|---|---|---|---|---|
| Pump | rate over 5 kg | rate per kg | temperature | Time |
| (rpm) | (g/min/5 kg) | (g/min/kg) | (° C.) | (min) |
| 16 | 30 | 6 | 30 | 0->5 |
| 22 | 40 | 8 | 28 | 5->76 |

A smoothing step was then carried out as follows, using the same solution as that used for the sugar-coating liquid.

TABLE 7

| | | | Time | | |
|---|---|---|---|---|---|
| | Amount of | | without | Time | |
| | solution | Spraying | spraying | without | |
| | nebulized | time | and | spraying | Total time |
| Number of | without | without | without | and with | of the |
| smoothing | drying | drying | drying air | drying air | smoothing |
| steps | air (g) | air (s) | (min) | (min) | step (min) |
| 3 | 50* | 35 | 2.5 | 0.5 | 11 |

*Pump speed: 50 rpm

### Test 2

The sugar-coating liquid had the following formulation:

TABLE 8

| | |
|---|---|
| Xylitol (XYLISORB ®90, ROQUETTE) | 58.1% |
| hydrolyzed and functionalized starch | 0.3% |
| (hydrolyzed hydroxypropylated starch | |
| LYCOAT ® RS720, ROQUETTE) (binder) | |
| Titanium dioxide (KRONOS) | 0.6% |
| Sum of ingredients other than the solvent | 59.0% |
| Demineralized water (solvent) | 41.0% |

The percentages are expressed by weight relative to the total weight of the sugar-coating liquid.

The sugar-coating liquids were prepared as in point 4. LYCOAT® RS720 was introduced in the form of a 25% solution.

The sugar-coating conditions were as follows:

TABLE 9

| | Spraying flow | Spraying flow | Core bed | |
|---|---|---|---|---|
| Pump | rate over 5 kg | rate per kg | temperature | Time |
| (rpm) | (g/min/5 kg) | (g/min/kg) | (° C.) | (min) |
| 16 | 30 | 6 | 30 | 0->5 |
| 22 | 40 | 8 | 28 | 5->72 |

A smoothing step was then carried out as in Test 1

### B. Stability

Stability tests were carried out on the sugar-coated products obtained according to section 4. and according to section 5., Test 1. To do this, the sugar-coated chewing gums were placed at 30° C. at 70% relative humidity without packaging for 3 weeks. The water uptakes were measured over time (in % mass gain by water absorption). The tests were also carried out under the following conditions: 40° C., 75% relative humidity, without packaging.

The two batches of chewing gums had the same water uptake profiles.

Stability tests were also carried out in a second step on other cores. These tests, not presented here, showed that the hydrolyzed and functionalized starch in accordance with the disclosure even made it possible to obtain better results than with gum arabic.

### Test 3

A test was also carried out wherein the sugar-coating liquid was produced by means of a ready-to-use powdery composition, having the following composition:

TABLE 12

| | |
|---|---|
| Xylitol (XYLISORB ®90, ROQUETTE) | 98.5% |
| Hydrolyzed and functionalized starch | 0.5% |
| (hydrolyzed hydroxypropylated starch | |
| LYCOAT ® RS720, ROQUETTE) (binder) | |
| Titanium dioxide (KRONOS) | 1.0% |

The percentages are expressed by weight relative to the total weight of the powdery composition.

The sugar-coating liquid had the following composition: 60% of this ready-to-use composition and 40% water.

Sugar-coating tests were carried out successfully by using this method.

The inventors also carried out tests with a xylitol of greater particle size (the xylitol of low particle size potentially forming clumps during storage). In these tests, XYLISORB® 90 (mean diameter approximately equal to 90 μm) was therefore replaced by XYLISORB® 300 (mean diameter of approximately 300 μm). Very good results were also obtained.

1. A sugar-coating method, comprising:
a step (a) of spraying a sugar-coating liquid comprising crystallizable materials onto a bed of moving cores, said cores being placed in a chamber equipped with a perforated rotary drum, and said spraying being performed by means of at least one compressed air nozzle;
at least one step (b) concomitant with step (a) of drying said sprayed sugar-coating liquid; and,
a step (c) of collecting the resulting sugar-coated solid forms;
wherein said sugar-coating liquid comprises a hydrolyzed and functionalized starch.

2. The sugar-coating method according to claim 1, wherein said crystallizable materials comprise xylitol.

3. The sugar-coating method according to claim 1, wherein said hydrolyzed and functionalized starch is functionalized with alkyl groups having 1 to 5 carbon atoms.

4. The sugar-coating method according to claim 1, wherein said hydrolyzed and functionalized starch is selected from hydroxypropylated starch, acetylated starch, hydroxyethyl starch, or a mixture thereof.

5. A sugar-coating liquid consisting of:
(a) 40 to 80% ingredients other than the solvent consisting of:
(a.1) 90.0 to 99.8% xylitol;
(a.2) 0.1 to 3.0% hydrolyzed and functionalized starch;
(a.3) 0.1 to 3.0% of an opacifier, and,
(a.4) 0.0 to 3.0% other ingredients;
the percentages (a.1) to (a.4) being expressed by weight relative to the total weight of ingredients other than the solvent, and their sum being equal to 100%; and
(b) 60 to 20% solvent;
the percentages (a) and (b) being expressed by weight relative to the total weight of the sugar-coating liquid, and their sum being equal to 100%.

6. A use of a sugar-coating liquid according to claim 5, for sugar-coating solid forms.

7. A sugar-coated solid form comprising at least one sugar-coating layer obtained from the sugar-coating liquid according to claim 5.

8. A powdery composition for the sugar-coating of solid forms, consisting of:
90.0 to 99.8% xylitol;
0.1 to 3.0% hydrolyzed and functionalized starch;
0.1 to 3.0% of an opacifier; and,
0.0 to 3.0% other ingredients;
the percentages being expressed by weight relative to the total weight of said powdery composition, and their sum being equal to 100%.

9. A use of a powdery composition according to claim 8, for sugar-coating solid forms.

10. A sugar-coated solid form comprising at least one sugar-coating layer obtained from the composition according to claim 8.
